(19) United States
(12) Patent Application Publication
(10) Pub. No.: US 2005/0192597 A1
(43) Pub. Date: Sep. 1, 2005

(54) Wound closure device

(75) Inventors: Manfred Boebel, Bauschlott (DE); Dieter Busche, Hamburg (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Richard Wolf GmbH

(21) Appl. No.: 10/887,046

(22) Filed: Jul. 8, 2004

(30) Foreign Application Priority Data
Jul. 8, 2003 (DE) 103 30 660.9

(51) Int. Cl. A61B 17/04

(52) U.S. Cl. 606/148

(57) ABSTRACT

A wound closure device is provided with at least one conical, proximal section, which tapers in the distal direction, and a tapered, distal section connecting thereto. A through-hole extends through the proximal section from the proximal end to a transition region precisely between the proximal and the distal sections and intersects the longitudinal axis of the wound closure device. A marking for the insertion depth is incorporated on the distal section distally spaced from the distal end of the through-hole.

### BACKGROUND OF THE INVENTION

The invention relates to a wound closure device.

A guide for suturing a wound, in particular a wound as occurs when using endoscopic apparatus, is known from U.S. Pat. No. 5,507,758. The guide comprises a cylindrical main section which at its proximal end comprises a disk-like projection. The cylindrical section of the guide is inserted so far into a puncture opening or a puncture channel until the peripheral projection at the proximal end bears as an abutment on the outer side of the tissue, i.e., on the surface of the skin. Two obliquely running through-holes are formed in the inside of the cylindrical section, so that instruments for suturing may be introduced from the proximal end-face of the guide into the through-holes. The through-holes are opened to the peripheral outer wall of the cylindrical section, such that instruments used for suturing enter the tissue through an exit hole and penetrate this tissue in order to suture.

The problem with these instruments, on the one hand, is the sealing effect which is achieved after the insertion into the puncture channel or the wound opening. In order to achieve an adequate sealing effect, it is necessary for the tissue surrounding the wound opening to bear on and over the whole periphery of the cylinder on its outer wall. With this guide known from U.S. Pat. No. 5,507,758 it is also a disadvantage that various guides need to be provided for the various patient anatomies, which have a distance of the distal exit holes to the projection on the proximal side which is adapted to the respective tissue thickness. Furthermore the arrangement of several through-holes in the inside of the guide may lead to a danger of confusion and thus to problems when suturing.

### BRIEF SUMMARY OF THE INVENTION

It is therefore an object of the present invention to create an improved wound closure device which ensures an improved sealing effect and may be universally applied to various patient anatomies.

According to the invention this object is achieved by a wound closure device comprising at least one conical, proximal section which tapers in a distal direction, a tapered, distal section connecting to the proximal section, exactly one through-hole extending through the proximal section from a proximal end to a transition region between the proximal section and the distal section. The through-hole intersects a longitudinal axis X of the wound closure device, and a marking for an insertion depth is incorporated on the distal section distally spaced from a distal end of the through-hole. Preferred embodiments may be deduced from the accompanying description and dependent claims.

The wound closure device according to the invention comprises at least one conical, proximal section, which tapers in the distal direction, and a tapered distal section which connects to the proximal section. The tapered distal section simplifies the introduction of the device into a puncture channel. The conical design of the proximal section effects an improved sealing of a pneumoperitoneum, since the tissue is pressed against the conical outer surfaces of the proximal section on account of the excess pressure in the body cavity. At the same time a projection on the proximal side does not need to be provided on the proximal section, against which the outer side of the tissue is pressed for sealing.

With the wound closure device according to the invention only the inner surface of the puncture channel is pressed on the outer surface of the proximal section. Exactly one through-hole runs through the proximal section from the proximal end to the transition region between the proximal and the distal section, and intersects the longitudinal axis of the wound closure device. The through-hole at the proximal end is essentially open towards the proximal end-face of the proximal section and at its distal end peripherally exits in the transition region between the proximal and distal section.

According to the invention only one through-hole needs to be provided in the wound closure device, which serves for the introduction of instruments for suturing a wound. The arrangement of only one through-hole has the advantage that with the use of the wound closure device there is no danger of confusion with several through-holes. After the thread has been introduced into the tissue through the through-hole, the instrument may be removed, and subsequently the wound closure device may be rotated about a defined angle in order then to again introduce an instrument through the through-hole and to retract the thread for suturing. In this manner the wound closure device according to the invention permits a simple suturing of a wound.

Moreover, according to the invention, a marking for the insertion depth is distally spaced from the distal end of the through-hole on the distal section. The incorporation of this marking permits the universal application of the wound closure device to different patient anatomies having various tissue thicknesses. For suturing, the wound closure device is introduced into the puncture channel to be sutured, up to the point where the marking penetrates the peritoneum under laparascopic viewing. If the marking appears in the abdomen, the distal end of the through-hole always lies at a defined position independently of the thickness of the tissue, for example of the abdominal wall. Thus, the same conditions are present, i.e., the same distance from the distal end of the through-hole, for example an exit location of the needle, to the free abdominal cavity. For this reason the wound closure device according to the invention may be applied universally to different patient anatomies with different thicknesses of abdominal walls. Despite this, a precise guiding of the needles relative to the tissue to be sutured, in particular to the fascia, may always be ensured in a simple manner.

Preferably the distal section too is conical, i.e., it is formed tapering in the distal direction. This design permits a simpler introduction of the wound closure device into a puncture channel, since the distal end of the distal section may be designed adequately slim, so that it may be easily inserted into the wound opening. On further insertion of the wound closure device, the conical shape of the distal section introduces the proximal section of the wound closure device, which has a larger diameter, into the puncture channel. Thus, a simple insertion of the wound closure device is possible without damaging the tissue.

The flank of the transition region between the proximal and the distal sections preferably runs in a conical manner, wherein it tapers in the distal direction. At the same time, the flank of the transition region is preferably inclined more steeply relative to the longitudinal axis of the wound closure device than the outer wall of the proximal and/or distal section. The chamfered or oblique design of the flank of the transition region has the advantage that the wound closure device on introduction into the puncture channel may not snag with the flank in the tissue. Alternatively, the distal section may connect directly to the distal end of the proximal section without a step or a flank inclined to the outer wall of the proximal and/or distal section being provided. The outer surface of the distal section may also merge directly into the peripheral surface of the proximal section, for example in the form of a continuous curvature. At the same time, the transition region may be designed in a concave or convex manner.

The proximal and the distal sections of the wound closure device according to the invention are preferably formed in a rotationally symmetrical manner. The complete wound closure device thus preferably comprises a circular cross section at each location, which permits a simple insertion into a wound opening. The cross sectional diameters change in the direction of the longitudinal axis of the wound closure device according to the conical or tapering shape of the proximal and, as the case may be, also of the distal section. A thickened handle is further preferably designed at the proximal end of the proximal section, which permits a simple gripping and thus an easy handling of the wound closure device. The handle may, for example, be designed spherically or hemispherically and have a larger diameter with respect to the proximal end of the proximal section, so that the wound closure device may be easily held.

It is furthermore preferred for at least one side of the handle to be flattened. This is preferably a side on the periphery of the handle with respect to the longitudinal axis of the wound closure direction. The flattened design prevents the wound closure device from rolling away and furthermore provides space for incorporating labeling (inscription).

The marking for the insertion depth on the distal section is preferably designed as a circumferential annular groove. This ensures that the marking may be surely recognized by an endoscope from each side, when the wound closure device has been introduced into the tissue. The annular groove is preferably coated with a color, for example it is designed blackened so that it may be able to be recognized in a clearer and simpler manner.

The through-hole is preferably countersunk at its proximal end, i.e., it is designed with a widened diameter. The introduction of instruments is simplified in this manner.

The marking is preferably spaced in the radial direction by about 5 mm from the distal end or the peripheral exit opening of the passage hole. This arrangement ensures that the distal exit hole of the through-hole, when the annular groove is visible in the abdomen, is positioned exactly such that a suture for suturing may be guided through the fascia by way of an instrument introduced into the through-hole. The arrangement thus permits a very accurate positioning of the wound closure device, by which a simple suturing becomes possible.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there is shown in the drawings an embodiment which is presently preferred. It should be understood, however, that the invention is not limited to the precise arrangements and instrumentalities shown. In the drawings:

FIG. 1 is a longitudinal side view of the wound closure device according to the invention; and

FIG. 2 is a longitudinal section through the wound closure device according to FIG. 1, rotated 90°.

### DETAILED DESCRIPTION OF THE INVENTION

The wound closure device according to the invention has a conical, distal section 2, which is designed as a slim cone, and a rounded tip 4 at its distal end. The distal section 2 serves for the simple introduction of the wound closure device into a wound opening or a puncture channel. A transition section 6 connects at the proximal end of the distal section 2, and this transition section likewise widens in the proximal direction. However, the flanks of the transition section have a stronger inclination with respect to the longitudinal axis X than the flanks of the distal section 2.

A proximal section 8, which is relatively short in the longitudinal direction X, connects to the transition section 6, and the diameter of this proximal section widens in the proximal direction proceeding from the transition section 6. At the same time, the conicity, i.e., the inclination of the peripheral surfaces of the proximal section 8 with respect to the longitudinal axis X, is considerably smaller that the inclination of the flanks of the transition section 6.

A handle 10, which is designed in an essentially hemispherical manner, connects to the proximal end of the proximal section 8, wherein the spherical outer surface of the handle 10 faces away from the proximal section 8. The handle 10 has a larger diameter than the proximal end of the proximal section 8, so that the handle 10 projects peripherally with respect to the proximal section 8 and forms a grip which may be easily grasped.

The complete wound closure device, comprising the handle 10, the proximal section 8, the transition region 6, and the distal section 12, is preferably formed as one piece. A peripheral annular groove 12 is formed in the distal section 2 spaced distally from the transition section 6 by the spacing S. The annular groove 12 serves as a marking for the insertion depth, as will be described further below.

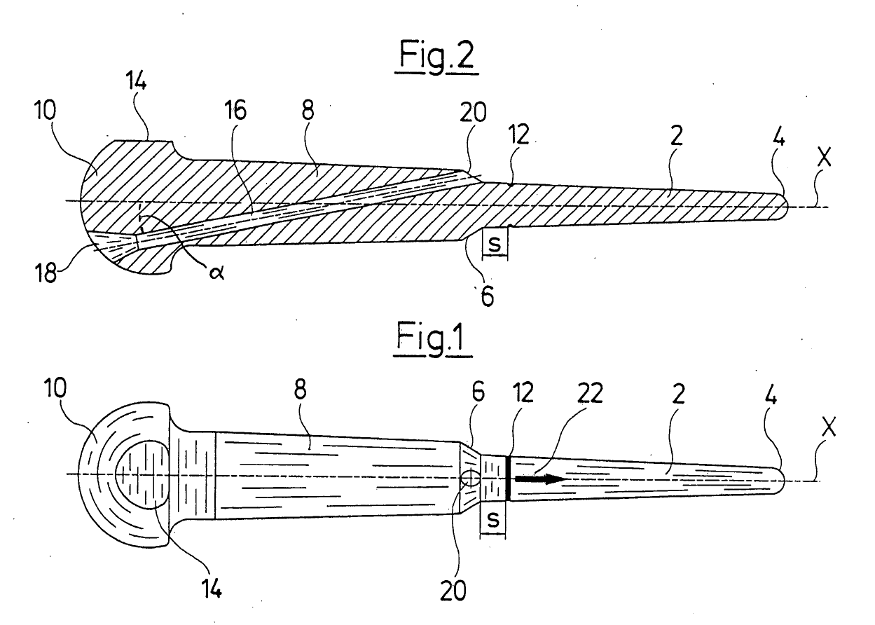

FIG. 2 show a sectioned view of the wound closure device according to FIG. 1 along the longitudinal axis X. Here, the position of the wound closure device is rotated by 90° about the longitudinal axis X with regard to FIG. 1. A side surface 14 of the handle 10 is designed flattened, so that the wound closure device may not roll away if it is placed on a table.

A through-hole 16 in the form of a bore extends inside of the proximal section 8. The through-hole 16 runs at an angle a to the longitudinal axis X, wherein it intersects the longitudinal axis X. The proximal end 18 of the through-hole 16 is opened to the proximal end of the handle 10. Here, the through-hole 16 at its proximal end 18 is countersunk, so that instruments may be easily introduced from the proximal end into the through-hole 16. The distal end 20 of the through-hole 16 is arranged in the region of the flank of the transition section 6, i.e., the through-hole 18 opens exactly in the transition section 6. The through-hole 16 extends obliquely through the proximal section in a manner such that the proximal end 18 and the distal end 20 of the through-hole 16 are situated on opposite sides of the longitudinal axis X. The length of the flank of the transition section 6 is selected such that the distal end 20 of the through-hole 16 borders directly onto the distal end of the proximal section and the proximal end of the distal section 2.

The use of the wound closure device according to the invention is described hereinafter. For suturing a puncture channel, for example following an endoscopic operation, the distal section 2 with its tip forward is inserted so far into the puncture channel, for example a trocar puncture opening, until the peripheral groove 12 penetrates the peritoneum and becomes visible in the abdomen under laparasocpic viewing. The annular groove 12 may additionally be highlighted from the surrounding material in a colored manner, e.g., it may be dyed black, in order to be more easily recognized.

In order to be able to position the wound closure device correctly in its angular position with respect to the longitudinal axis X, a marking 22, in the present example in the form of an arrow, is incorporated on the distal section 2 on the distal side of the annular groove 12. The marking 22 is located on the periphery of the distal section 2 at the same angular position as the distal end 20 of the through-hole 16. Thus under endoscopic control one may exactly ascertain in which position with respect to the longitudinal axis X the distal end 20 of the through-hole 16 is located, even if the distal end 20 itself does not enter the abdomen and thus is not endoscopically visible. However, since the distal section 2 in the fully inserted condition in the abdomen is completely visible, the marking 22 may be easily recognized under endoscopic control, and thus the angular position of the wound closure device may be selected as desired.

Upon introduction, the shape of the distal section 2 and of the proximal section 8, which widens in the proximal direction, leads to the result that the wound closure device is introduced into the puncture channel, and this is widened to such an extent that the walls of the puncture channel come to sealingly bear on the conical peripheral surface of the proximal section 8. Since a pneumoperitoneum prevails in the inside of the body cavity, the tissue, for example the abdominal wall, in the region of the puncture channel is pressed against the conical peripheral wall of the proximal section 8 by way of the excess pressure inside the body cavity, so that a good sealing effect is achieved.

When the wound closure device is inserted so far that the annular groove 12 is visible in the abdomen, a suture is inserted through the through-hole 16 into the abdomen with a suitable needle or a sharp forceps. Since the wound closure device may only be introduced into the abdominal wall to such an extent that the annular groove 12 just becomes visible, the needle or forceps which guides the suture penetrates exactly through the fascia into the abdomen. In order to achieve this, the distance s between the annular groove 12 and the distal end 20 of the through-hole 16 must be selected accordingly, preferably about 5 mm.

The suture is then released under endoscopic control, and the needle is retracted from the through-hole 16 in the proximal direction until the tip of the needle no longer projects out of the distal end 20 of the through-hole 16. For this, a marking may be provided in the region of the proximal end of the needle or needle forceps, which is spaced from the tip of the needle by an amount which corresponds at least to the length of the through-hole 16, so that it may be easily seen from the region of the handle 10 whether the tip of the needle projects out of the distal end of the through-hole 16.

Subsequently, the needle or the forceps is moved further in the distal direction in the through-hole 16, so that the needle tip again exits from the distal end 20 of the through-hole 16. At the same time, the fascia is again punctured by the tip of the needle or forceps, and the suture may be taken up again. On leading the forceps or needle back in the proximal direction, the suture is again transported out of the body. After the removal of the wound closure device from the puncture channel, the ends of the suture may then be knotted, and the fascia may be closed separately in a loose manner. A pneumoperitoneum may be created again after this closure, and the closure location may be controlled.

The diameter of the wound closure device, in particular the diameter of the proximal section 8 is selected such that the wound closure device is adapted to a certain trocar sleeve diameter. The size of the proximal section 8 is always selected such that a sealing of the abdominal wall on the peripheral wall of the proximal section 8 is effected.

The exact shape of the stitch or suture is determined by the extent and the number of the individual rotating steps of the wound closure device about its longitudinal axis X. As described above, one may also place several stitches depending on the number of rotation steps, in order to close the wound opening.

On account of the marking of the wound closure device for penetration depth being arranged at a fixed predefined distance s to the distal end 20 of the through-hole 16, the wound closure device may be used independently of the respective thickness of the abdominal wall of the patient. The proximal end of the wound closure device then projects outward by a different amount depending on the thickness of the abdominal wall. The distal end 10 of the through hole 16, which serves for the exact positioning of the needle and thus of the puncturing, however always lies at a predefined location relative to the free abdominal cavity. One may thus always place the required suture at a predefined depth of the abdominal wall.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A wound closure device comprising at least one conical, proximal section (8) which tapers in a distal direction, a tapered, distal section (2) connecting to the proximal section 8, exactly one through-hole (16) extending through the proximal section (8) from a proximal end to a transition region (6) between the proximal section (8) and the distal section (2), wherein the through-hole (16) intersects a longitudinal axis (X) of the wound closure device, and a marking (12) for an insertion depth being incorporated on the distal section (2) distally spaced from a distal end (20) of the through-hole (16).

2. The wound closure device according to claim 1, wherein the distal section (2) is formed conically, tapering in the distal direction.

3. The wound closure device according to claim 1, wherein a flank of the transition region (6) between the proximal (8) and the distal section (2) is formed conically, tapering in the distal direction.

4. The wound closure device according to claim 1, wherein the proximal section (8) and the distal section (2) are designed in a rotationally symmetrical manner.

5. The wound closure device according to claim 1, wherein a thickened handle (10) is formed at a proximal end of the proximal section (8).

6. The wound closure device according to claim 5, wherein the handle (10) is formed flattened at least on one side.

7. The wound closure device according to claim 1, wherein the marking (12) is designed as a peripheral annular groove.

8. The wound closure device according to claim 1, wherein the through-hole (16) is countersunk at its proximal end (18).

9. The wound closure device according to claim 1, wherein the marking (12) is spaced from the distal end (20) of the through-hole by about 5 mm in the distal direction.
